(19) United States
(12) Patent Application Publication
(10) Pub. No.: US 2021/0038664 A1
(43) Pub. Date: Feb. 11, 2021

(54) FORMULATION AND METHOD OF USE

(71) Applicant: ATP Institute Pty Ltd, Queensland (AU)

(72) Inventor: Matthew Legge, Queensland (AU)

(21) Appl. No.: 16/968,518

(22) PCT Filed: Feb. 8, 2019
(86) PCT No.: PCT/AU2019/050097

§ 371 Date: Aug. 7, 2020

(30) Foreign Application Priority Data
Feb. 9, 2018 (AU) 2018900407

(51) Int. Cl. A61K 36/074; A61K 36/324; A61K 36/9066; A61K 36/282; A61K 36/31; A61K 47/46; A61K 47/22; A61K 9/00; A61K 9/48; A61K 31/05; A23L 7/126; A23L 33/105; A23L 33/15; A23L 25/00; A23L 33/21

(52) U.S. Cl. CPC A61K 36/074; A23L 33/21; A61K 36/9066; A61K 36/282; A61K 36/31; A61K 47/46; A61K 47/22; A61K 9/0056; A61K 9/4875; A61K 31/05; A23L 7/126; A23L 33/105; A23L 33/15; A23L 25/00; A61K 36/324

(57) ABSTRACT

Provided herein is an orally administrable formulation for promoting gastrointestinal health, the formulation including a plurality of plant-based polyphenol sources and/or one or more components or derivatives thereof together with one or more of a fibre source, a sweetening agent and/or a flavouring agent as well as methods of making same. Methods of using the orally administrable formulation in promoting gastrointestinal health, modulating microbial flora in a subject's gastrointestinal tract and the treatment of a gastrointestinal disease, disorder or condition are also provided.

### TECHNICAL FIELD

THIS INVENTION relates to formulations for the promotion of gastrointestinal health and methods of treatment and/or use thereof. The formulations are useful for a variety of applications, such as modulating a subject's gut microbiome and/or the treatment of diseases, disorders or conditions characterised, at least in part by poor gastrointestinal health, including inflammatory bowel disease (IBD).

### BACKGROUND

Our modern dietary habits have evolved over time, such that our food sources and diets have changed based on range of factors, such as lower micronutrient and fibre levels, higher sugar levels, intensive farming techniques, species selection and genetically modified food sources, no regular fasting, food cooking, processing and storage techniques, consuming less fermented foods and taking more supplements or fortified foods. This has led to an increase in the incidence of poor gastrointestinal health owing to significant changes in a subject's microbiome as a result of this modern diet.

Accordingly, there exists a need for improved formulations or supplements for modifying the gut microbiome and in doing so further promote improved gut health and/or prevent or treat diseases, disorders or conditions associated with poor gut health.

### SUMMARY

The present invention is directed to formulations and methods for promoting gastrointestinal health and treating and/or preventing gastrointestinal diseases, disorders or conditions.

In a broad form, the invention relates to orally administrable formulations comprising a plurality of plant-based polyphenol sources and one or more of a sweetening agent, a flavouring agent and a fibre source for use in promoting gastrointestinal health and/or the treatment of a gastrointestinal disease, disorder or condition, such as inflammatory bowel disease, irritable bowel syndrome and dysbiosis. Suitably, the formulation is substantially free of probiotic microorganisms.

In a first aspect the invention provides an orally administrable formulation for promoting gastrointestinal health comprising, consisting or consisting essentially of:
(i) a plurality of plant-based polyphenol sources and/or one or more components or derivatives thereof; and
(ii) one or more of a fibre source, a sweetening agent and/or a flavouring agent.

Broadly, the plant-based polyphenols may be obtainable or derivable from any plant or plant part, inclusive of the whole plant. Suitably, the plant-based polyphenol source is selected from the group consisting of Raphanus sativus (daikon radish), Brassica oleracea spp., Hordeum vulgare (barley), Euterpe oleracea (acai), Larix spp. heartwood, Hibiscus sabdarifa (rosella), Theobroma cacao, Prunus serotina (black cherry), Myristica fragrans (nutmeg), Zingiber officinale (ginger), Allium cepa (onions), Allium sativum (garlic), Cinnamomum verum or other cinnamon species, Musa spp. (green banana), Schisandra chinensis, Punica granatum (pomegranate), Rosmarinus officinalis (rosemary), Malus spp. (apple), Vaccinium spp. (cranberry), berberine, Ilex paraguariensis (yerba mate), Coffea spp., Ganoderma spp., Lentinula edodes (shiitake), Curcuma longa (turmeric), Boswellia spp. (frankincense), Artemisia spp., Matricaria chamomilla (chamomile), Rumex crispus (yellow dock), Mahonia aquifolium (oregon grape), Hydrastis canadensis (goldenseal), Calendula spp., Vicia faba (faba bean), Vigna radiata (mungbean), Cicer arietinum (chick pea), Pseudowintera colorata, graviola (soursop), Pimpinella anisum (anise), Lycium barbarum (goji berry), Lycium chinense (goji berry), Prunus spp. (cherry), Beta vulgaris (beetroot), Commiphora spp. (myrrh), Salvadora persica (Israeli mustard) a nut, a herb, a fruit extract, a vegetable extract and any combination thereof.

In certain embodiments, one or more of the plant-based polyphenol sources and/or one or more components or derivatives thereof, includes one or more of an extract thereof, a skin portion, a peel portion, a seed portion, a leaf portion, a sprout portion, a juice portion, a husk portion, a root portion and a pulp portion.

The fibre source is suitably selected from the group consisting of a konjac fibre source or flour, a psyllium fibre source, such as psyllium husk, a mucopolysaccharide, such as slippery elm bark, inulin, a sugarcane fibre source (e.g., Saccharum officinarum), a chick pea fibre source, a green banana fibre source, a faba bean fibre source, a mung bean fibre source, a nut meal, a legume meal, a seed meal, a grain flour, a husk flour, a bran flour and any combination thereof.

In one particular embodiment, the formulation is substantially free of probiotic microorganisms.

In one embodiment, the formulation comprises from about 40 wt % to about 95 wt % of the plant-based polyphenol sources.

In one embodiment, the formulation comprises from about 1 wt % to about 25 wt % of the fibre source.

In one embodiment, the formulation comprises from about 0.1 wt % to about 5 wt % of the sweetening agent.

In one embodiment, the formulation comprises from about 0.1 wt % to about 5 wt % of the flavouring agent.

Suitably, the formulation of the present aspect further comprises one or more vitamins and/or minerals.

In particular embodiments, the formulation is in the form of a food product.

In a second aspect, the invention provides a method of producing the orally administrable formulation according to the first aspect, including the step of combining the plurality of plant-based polyphenol sources and/or one or more components or derivatives thereof with the fibre source, the sweetening agent and/or the flavouring agent to thereby produce the orally administrable formulation

In a third aspect, the invention provides an orally administrable formulation produced according to the method of second aspect.

In a fourth aspect, the invention provides an orally administrable formulation according to any one of the first and third aspects, for use in:

(i) promoting gastrointestinal health;

(ii) modulating microbial flora in at least a portion of a gastrointestinal tract; and/or

(iii) the therapeutic and/or prophylactic treatment of a gastrointestinal disease, disorder or condition;

in a subject.

In a fifth aspect, the invention provides a method of promoting gastrointestinal health in a subject, said method including the step of administering to said subject a therapeutically effective amount of the orally administrable formulation according to any one of the first and third aspects, to thereby promote gastrointestinal health in the subject.

In one embodiment, administration of the orally administrable formulation modulates one or more species or genera of microbial flora in at least a portion of a gastrointestinal tract of the subject.

In a sixth aspect, the invention provides a method of modulating microbial flora in at least a portion of a gastrointestinal tract of a subject, said method including the step of administering to the subject the orally administrable formulation according to any one of the first and third aspects in an amount effective to achieve said modulation.

In a seventh aspect, the invention provides a method of treating and/or preventing a gastrointestinal disease, disorder or condition in a subject, said method including the step of administering to said subject a therapeutically effective amount of the orally administrable formulation according to any one of the first and third aspects, to thereby treat and/or prevent said gastrointestinal disease, disorder or condition in the subject.

Suitably, the gastrointestinal disease, disorder or condition is selected from the group consisting of candidiasis, celiac disease, Crohn's disease, diarrhoea, constipation, ulcerative colitis, food allergy, food intolerance, inflammatory bowel disease (IBD), irritable bowel syndrome (IBS), intestinal dysbiosis, metabolic syndrome, ulcers, digestion disorders, malabsorption syndromes, gastritis, enteritis, gastroesophageal reflux, eosinophilic gastroenteritis, infectious diarrhoea, collagenous colitis and lymphocytic colitis, diversion colitis, indeterminate colitis, nonsteroidal anti-inflammatory drug enteropathy, non-celiac gluten sensitivity, coeliac disease, acute self-limiting colitis, amoebic colitis, schistosomiasis, colon cancer, intestinal tuberculosis and any combination thereof.

In one embodiment, administration of the orally administrable formulation modulates one or more species or genera of microbial flora in at least a portion of a gastrointestinal tract of the subject.

Referring to the method of the fifth, sixth and seventh aspects, the microbial flora suitably include one or more microorganisms of a genus selected from the group consisting of Achromobacter, Acidaminococcus, Acinetobacter, Actinomyces, Aeromonas, Aggregatibacter, Akkermansia, Alcaligenes, Alistipes, Anaerobiospirillum, Arachnia, Bacillus, Bacteroides, Bacterionema, Bifidobacterium, Buchnera, Butyriviberio, Campylobacter, Candida Capnocytophaga, Citrobacter, Clostridium, Corynebacterium, Eikenella, Enterobacter, Enterococcus, Escherichia, Eubacterium, Flavobacterium, Fusobacterium, Gordonia, Haemophilus, Lactobacillus, Leptotrichia, Methanobrevibacter, Morganella, Mycobacteria, Mycoplasma, Micrococcus, Neisseria, Parabacteroides, Peptococcus, Peptostreptococcus, Plesiomonas, Porphyromonas, Prevotella, Propionibacterium, Providencia, Pseudomonas, Ruminococcus, Rothia, Sarcina, Staphylococcus, Streptococcus, Torulopsis, Treponema, Veillonella, Vibrio, Wolinella, Yersinia and any combination thereof.

With respect to the invention of the fourth, fifth, sixth and seventh aspects, the subject is suitably a human.

Throughout this specification, unless otherwise indicated, “comprise”, “comprises” and “comprising” are used inclusively rather than exclusively, so that a stated integer or group of integers may include one or more other non-stated integers or groups of integers. Conversely, the terms “consist”, “consists” and “consisting” are used exclusively, such that a stated integer or group of integers are required or mandatory, and no other integers may be present.

The phrase “consisting essentially of” indicates that a stated integer or group of integers are required or mandatory, but that other elements that do not interfere with or contribute to the activity or action of the stated integer or group of integers are optional.

As used in this specification the indefinite articles “a” and “an” may refer to one entity or a plurality of entities and are not to be read or understood as being limited to a single entity. For example, “a” subject includes one subject, one or more subjects or a plurality of subjects

### DETAILED DESCRIPTION

The present inventors have created an improved orally administrable formulation or “modbiotic” which, when administered to a subject is designed to promote gastrointestinal health and/or the treatment of a gastrointestinal disease, disorder or condition. In this regard, the formulations described herein, which include a plurality of plant-based polyphenol sources and one or more of a sweetening agent, a flavouring agent and a fibre source, are designed to modulate the microbial flora of a subject's a gastrointestinal tract upon administration thereto. Suitably, the formulation is substantially free of probiotic microorganisms.

In a first aspect the invention provides an orally administrable formulation for promoting gastrointestinal health comprising, consisting or consisting essentially of:
(iii) a plurality of plant-based polyphenol sources and/or one or more components or derivatives thereof; and
(iv) one or more of a fibre source, a sweetening agent and a flavouring agent.

For purposes of this invention, the term “plant-based polyphenol source” generally refers to any polyphenol source that is, or can be, obtained or derived from plants or plant parts, including synthetically manufactured polyphenols. Natural polyphenols, however, are preferred. It will be appreciated that this term can refer to, for example, fruit-based, vegetable-based, grass-based, herb-based, root-based, weed-based, nut-based and/or seed-based polyphenol sources.

As generally used herein, the term “polyphenol” refers to a class of compounds which typically include a plurality of hydroxyl groups attached to one or more aromatic groups. These aromatic groups can be monocyclic (for example as in benzene), bicyclic (for example as in naphthalene), or polycyclic (for example as in anthracene). The polyphenol may be natural, synthetic or a mixture thereof. Exemplary polyphenols, albeit without limitation thereto, include dopamine, adrenaline, noradrenaline, salbutamol, curcumin and/or its derivatives, yakuchinone A, yakuchinone B, rosmarinic acid, paradol, hydroxytyrosol, silymarin, coumarin, esculetin, escopoletin, lignans (including sesamol, sesamin, sesamolin), carnosol, oleuropein, uric acid, ubiquinol, thymolphtaleine, phenolphthalein, carthamin, polyporic acid, atromentin, bovichinon-3, grevillin A, grevillin B, grevillin D, alkannin, shikonin, alizarin, purpurin, pseudopurpurin, purpuroxanthin, rubiadin, munjistin, chinizarin, morindon, emodin, aloe-emodin, rhein, chrysophanol, kermesic acid, flavokermesic acid, carminic acid, ellagic acid, spinochrome C, spinochrome D, spinochrome E, echinochrome A, red alkannin, hypericin, chrysophanic acid, betanidin, isobetanidin, pyrocatechol, pyrogallol, gallic acid and/or its esters, caftaric acid, chlorogenic acid, elonolic acid, protocatechuic acid, syringic acid, gentisic acid, caffeic acid, hops acids (including humulone, lupulone, colupulone), magnolol, honokiol, biphenols, di-resorcinol sulphide, bithionol, bromochlorophen, dioxybenzone, bisoctrizole, bemotrizinol, resveratrol, tannins (such as tannic acid), phenylpropanoids, flavonoids (including flavones (such as luteolin, apigenin, baicalin, tangeritin), flavonols (such as quercetin, galantin, kaempferol, myricetin, fisetin, isorhamnetin, pachypodol, rhamnazin, rutin, hydroxyethylrutosides), flavanones (such as hesperetin, naringenin, eriodictyol), 3-hydroxyflavanones (such as dihydroquercetin, dihydrokaempferol), isoflavones (such as genistein, daidzein, glycitein), neoflavonoids, flavan-3-ols (such as catechins, theaflavins), and anthocyanidins (such as cyanidin, delphinidin, malvidin, pelargonidin, peonidin, petunidin)), inclusive of variants or derivatives thereof. In one preferred embodiment, the polyphenol is selected from the group consisting of quercetin, kaempferol, luteolin, baicalin, ellagic acid, rosmarinic acid, naringenin and any combination thereof.

With respect to the formulation of the present aspect, the plant-based polyphenol source may include one or more polyphenols, such as those hereinbefore described, in a wt % concentration of about 0.1% to about 99% or any range therein such as, but not limited to, about 1% to about 35% or about 2% to about 20%. In particular embodiments the polyphenol is present at a wt % concentration of about 0.1, 0.2, 0.3, 0.4, 0.5, 0.6, 0.7, 0.8, 0.9, 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and any range therein. In particularly preferred embodiments, the plant-based polyphenol source may include one or more polyphenols present at a wt % concentration of about 1% to about 30%.

In particular embodiments, the plant-based polyphenol source is or comprises a polyphenol-rich extract or concentrate. It will be appreciated that such polyphenol-rich extracts or concentrates may be in any form, including liquid and solid forms. In particular embodiments, the polyphenol-rich extract or concentrate comprises at least about 5 wt %, preferably at least about 10 wt % and more preferably at least about 20 wt % of polyphenols.

The formulation of the present aspect suitably includes the plurality of plant-based polyphenol sources in a wt % concentration of about 20% to about 99% or any range therein such as, but not limited to, about 25% to about 95% or about 40% to about 80%. In particular embodiments the plurality of plant-based polyphenol sources are present in the present formulation at a wt % concentration of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and any range therein. In particularly preferred embodiments, the formulation comprises from about 40 wt % to about 95 wt % of the plurality of plant-based polyphenol sources.

In view of the above, the formulation of the present aspect preferably comprises one or more polyphenols at a wt % concentration of at least about 1% (e.g., 1, 2, 3, 4, 5, 7.5, 10, 15, 20, 25, 30, 35, 40, 45, 50 etc wt %), more preferably at least about 5 wt % and even more preferably at least about 10 wt %.

The terms “component” and “derivative” refer to any product/s which may be derived from plant-based polyphenol sources using a downstream processing technique or techniques (e.g. a series of techniques) known in the art, such as extraction and purification techniques. Accordingly, in certain embodiments, one or more of the plant-based polyphenol sources and/or one or more components or derivatives thereof, include one or more of an extract thereof, a skin portion, a peel portion, a seed portion, a leaf portion, a sprout portion, a juice portion, a husk portion, a root portion and a pulp portion.

Suitably, the plant-based polyphenol source is selected from the group consisting of Raphanus sativus (daikon radish), Brassica oleracea spp., Hordeum vulgare (barley), Euterpe oleracea (acai), Larix spp. heartwood, Hibiscus sabdarifa (rosella), Theobroma cacao, Prunus serotina (black cherry), Myristica fragrans (nutmeg), Zingiber officinale (ginger), Allium cepa (onions), Allium sativum (garlic), Cinnamomum verum or other cinnamon species, Musa spp. (green banana), Schisandra chinensis, Punica granatum (pomegranate), Rosmarinus officinalis (rosemary), Malus spp. (apple), Vaccinium spp. (cranberry), berberine, Ilex paraguariensis (yerba mate), Coffea spp., Ganoderma spp., Lentinula edodes (shiitake), Curcuma longa (turmeric), Boswellia spp. (frankincense), Artemisia spp., Matricaria chamomilla (chamomile), Rumex crispus (yellow dock), Mahonia aquifolium (oregon grape), Hydrastis canadensis (goldenseal), Calendula spp., Vicia faba (faba bean), Vigna radiata (mungbean), Cicer arietinum (chick pea), Pseudowintera colorata, graviola (soursop), Pimpinella anisum (anise), Lycium barbarum (goji berry), Lycium chinense (goji berry), Prunus spp. (cherry), Beta vulgaris (beetroot), Commiphora spp. (myrrh), Salvadora persica (Israeli mustard) a nut, a herb, a fruit extract, a vegetable extract and any combination thereof. In one preferred embodiment, the plant-based polyphenol source is selected from the group consisting of pomegranate, rosemary, rosella, cranberry, turmeric, myrrh, brassica spp., ginger, cinnamon, yerba mate, frankincense, berberine and any combination thereof.

The fibre source may include any natural or non-natural fibre source as are known in the art. Generally, fibre is a type of carbohydrate that forms the indigestible parts or component of plant-based foods, such as vegetables, fruits, grains, beans and legumes. It typically has two main components, soluble fibre and insoluble fibre, and may include non-starch polysaccharides such as arabinoxylans, cellulose, and other plant components such as resistant starch, resistant dextrins, inulin, lignin, chitins, pectins, beta-glucans, and oligosaccharides. In particular embodiments, the fibre source is selected from the group consisting of a konjac fibre source or flour, a psyllium fibre source, such as psyllium husk, a mucopolysaccharide, such as slippery elm bark, inulin, a sugarcane fibre source (e.g., Saccharum officinarum), a chick pea fibre source, a green banana fibre source, a faba bean fibre source, a mung bean fibre source, a nut meal, a legume meal, a seed meal, a grain flour, a husk flour, a bran flour and any combination thereof.

The formulation of the present aspect suitably includes the fibre source in a wt % concentration of about 1% to about 25% or any range therein such as, but not limited to, about 2% to about 20% or about 5% to about 15%. In particular embodiments the fibre source is present in the formulation at a wt % concentration of about 1, 1.25, 1.5, 1.75, 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75, 12, 12.25, 12.5, 12.75, 13, 13.25, 13.5, 13.75, 14, 14.25, 14.5, 14.75, 15, 15.25, 15.5, 15.75, 16, 16.25, 16.5, 16.75, 17, 17.25, 17.5, 17.75, 18, 18.25, 18.5, 18.75, 19, 19.25, 19.5, 19.75, 20, 21, 22, 23, 24, 25 and any range therein. In particularly preferred embodiments, the formulation comprises from about 5 wt % to about 15 wt % of the fibre source.

As generally used herein, the term “sweetening agent” refers to natural or artificial compounds used to increase the sweetness of the present formulation. It will be appreciated that sweetening agents include carbohydrate sweetening agents, (i.e., sugars and other carbohydrate sweetening agents) and non-carbohydrate sweetening agents. As used here the term “sugar” refers to sucrose and the constituents of sucrose (e.g., glucose and/or fructose, sugar syrup, malt syrup, maple syrup, starch syrup, glucose syrup, high-fructose syrups such as high-fructose corn syrup, honey, molasses) and other carbohydrates that can be used as sweetening agents or a source of these. The term “other carbohydrate sweetening agents” refers to, for example, sugar alcohols, such as erythritol, xylitol, maltitol, lactitol and sorbitol. Suitable examples of the non-carbohydrate sweetening agents include e.g. stevia, thaumatin, aspartame, acesulfame potassium (Ace-K), saccharin, cyclamates and sucralose. Accordingly, the sweetener may include a low calorie sweetener, a natural sweetener, a non-nutritive sweetener and/or an artificial sweetener. Additionally, the sweetener may be naturally or synthetically derived. Suitably, the sweetening agent or combination of sweetening agents are present at a concentration of about 0.01 to about 20 wt %, more preferably about 0.05 to about 10 wt % or even more preferably about 0.1 to about 2 wt %.

The flavouring agents may be any natural or non-natural substance which adds or enhances flavour. The flavouring agents may also comprise natural or non-natural stabilizers, anti-caking and/or flow agents. Flavouring agents are typically comprised of a flavoured substance(s) and complexes manufactured or extracted from nature in liquid or powdered form to impart a particular flavour into a product. Excipients are added to preserve, stabilize and maintain form and colour. Typical excipients may include flow agents, anticaking agents, antioxidants, including but not exclusively, maltodextrin, gum acacia, tapioca starch, propylene glycol and triacetin. Suitably, the flavouring agent or combination of flavouring agents are present at a concentration of about 0.01 to about 20 wt %, more preferably about 0.05 to about 10 wt % or even more preferably about 0.1 to about 2 wt %.

In particular embodiments, the formulation of the present aspect further includes a filler, such as those known in the art. It will be appreciated that a filler is an ingredient added to provide bulk or some other non-nutritive purpose to a composition or formulation.

Suitably, the formulation of the present aspect further comprises one or more vitamins and/or minerals. Exemplary vitamins include vitamin A (e.g., vitamin Ai retinol, axerophthol, α-carotene, β-carotene, γ-carotene), B vitamins (e.g., B₁ vitamins including: thiamin, aneurin, thiamine, pyrophosphate, cocarboxylase; B₂ vitamins including riboflavin, vitamin G, lactoflavin, hepatoflavin, ovoflavin, verdoflavin, riboflavin mononucleotide, FMN, riboflavin dinucleotide, FAD), Vitamin C (ascorbic acid, antiscorbutic vitamin, dehydroascorbic acid), Vitamin D (antirachitic vitamin, vitamin D₂, D₃, cholecalciferol, etc), Vitamin E, Vitamin K, and the like. Nonlimiting examples of minerals include selenium, zinc, magnesium, calcium, iron, manganese, copper, chromium, phosphorous, iodine, potassium and molybdenum.

In particular embodiments, the formulation is in the form of a food product. Examples of suitable food products include a bar, such as a cereal or protein bar, a breakfast cereal, such as granola, a cracker, a biscuit and a snack, such as a snack chip. The preparation of these products is well known to the skilled person and does not require further detail here.

As used herein, “% concentration”, unless otherwise specified, may refer to percent weight/volume (w/v), percent weight/weight (w/w) or percent volume/volume (v/v) of a particular ingredient within the formulation as applicable.

Preferably, the formulation is substantially free of probiotic microorganisms and/or agents. By “substantially free” is meant that the orally administrable formulation is either completely free of any probiotic microorganisms or it is free to the extent that any probiotic microorganisms which may be present are sufficiently small that their presence does not adversely affect the ability of the formulation described herein to modify a subject's microbiome thereby promoting gastrointestinal health and/or preventing or treating a gastrointestinal disease, disorder or condition (e.g., preferably less than 0.5 wt % and more preferably less than 0.1 wt %). In this regard, supplementing a subject's diet with probiotics, such as Lactobacillus spp., can negatively modify or cause microflora imbalances within a subject's microbiome by promoting overgrowth of pathogenic microflora populations (e.g., Firmicute bacteria).

In a further aspect, the invention provides a method of producing the orally administrable formulation according to the aforementioned aspect, including the step of combining the plurality of plant-based polyphenol sources and/or one or more components or derivatives thereof with the fibre source, the sweetening agent and/or the flavouring agent to thereby produce the orally administrable formulation.

The method of the present aspect suitably preserves the ability of the plurality of plant-based polyphenol sources to promote gastrointestinal health. By way of example, preserving these properties may be achieved by avoiding exposing the formulation to excessive heat. As such, combining the ingredients of the orally administrable formulation at room temperature may improve the efficacy thereof.

In a related aspect, the invention provides an orally administrable formulation produced according to the aforementioned aspect.

In another aspect, the invention provides an orally administrable formulation described herein, for use in:

(i) promoting gastrointestinal health;

(ii) modulating microbial flora in at least a portion of a gastrointestinal tract;

and/or,

(iii) the therapeutic and/or prophylactic treatment of a gastrointestinal disease, disorder or condition;

in a subject.

Microorganisms that inhabit the gastrointestinal tract can influence human health through the production of metabolic by-products and short chain fatty acids, by stimulation of host immune response, or through other mechanisms. The gastrointestinal tract typically contains beneficial microflora which aid in gastrointestinal tract function and provide other health benefits. However, pathogenic or putrefactive microorganisms can also inhabit and colonize the gastrointestinal tract. There is a constant dynamic between beneficial and pathogenic flora populations in the gastrointestinal tract, and the latter can become dominant under certain conditions, such as stress, illness, and changes in diet or physiologic alterations in the gastrointestinal tract.

Without being bound by any theory, it is believed that feeding carbohydrates with insufficient fibre and polyphenols results in an overgrowth of pathogenic microflora, such as Firmicute bacteria, within a subject's gut microbiome. Additionally, supplementation with probiotics, and in particular Lactobacillus spp., may further stimulate Firmicute bacterial overgrowth. In nature, sugars from fruits, vegetables, cereals, grains, nuts and seeds would be supplied together with antimicrobial polyphenols to positively modulate the gut microbiome by manipulating the ratios between the respective strains therein and controlling or limiting any potential overgrowth thereof. Accordingly, plant-based polyphenol sources can play important roles in maintaining a balanced healthy intestinal microflora, and thereby promote gastrointestinal health.

In particular embodiments, the microbial flora to be modulated include one or more microorganisms of a genus selected from the group consisting of Achromobacter, Acidaminococcus (e.g., Acidaminococcus fermentans), Acinetobacter (e.g., Acinetobacter calcoaceticus), Actinomyces (e.g., Actinomyces viscosus, Actinomyces naeslundii), Aeromonas, Aggregatibacter (e.g., Aggregatibacter actinomycetemcomitans), Akkermansia, Alcaligenes (e.g., Alcaligenes faecalis), Alistipes, Anaerobiospirillum, Arachnia (e.g., Arachnia propionica), Bacillus, Bacteroides (e.g., Bacteroides gingivalis, Bacteroides fragilis, Bacteroides intermedius, Bacteroides gingivalis, Bacteroides fragilis, Bacteroides intermedius, Bacteroides melaninogenicus, Bacteroides pneumosintes), Bacterionema (e.g., Bacterionema matruchotii), Bifidobacterium, Buchnera (e.g., Buchnera aphidicola), Butyriviberio (e.g., Butyriviberio fibrosolvens), Campylobacter (e.g., Campylobacter coli, Campylobacter sputorum, Campylobacter upsaliensis), Candida (e.g., Candida albicans) Capnocytophaga, Citrobacter (e.g., Citrobacter freundii), Clostridium (e.g., Clostridium difficile, Clostridium sordellii), Corynebacterium, Eikenella (e.g., Eikenella corrodens), Enterobacter (e.g., Enterobacter cloacae), Enterococcus (e.g., Enterococcus faecalis, Enterococcus faecium), Escherichia (e.g., Escherichia coli), Eubacterium, Flavobacterium, Fusobacterium (e.g., Fusobacterium nucleatum), Gordonia, Haemophilus (e.g., Haemophilus parainfluenzae, Haemophilus paraphrophilus), Lactobacillus, Leptotrichia (e.g., Leptotrichia buccalis), Methanobrevibacter (e.g., Methanobrevibacter smithii), Morganella (e.g., Morganella morganii), Mycobacteria (e.g., Mycobacterium chelonae), Mycoplasma, Micrococcus, Neisseria (e.g., Neisseria sicca), Parabacteroides, Peptococcus, Peptostreptococcus, Plesiomonas (e.g., Plesiomonas shigelloides), Porphyromonas (e.g., Porphyromonas gingivalis), Prevotella, Propionibacterium (e.g., Propionibacterium acnes), Providencia, Pseudomonas (e.g., Pseudomonas aeruginosa), Ruminococcus (e.g., Ruminococcus bromii), Rothia (e.g., Rothia dentocariosa), Sarcina, Staphylococcus (e.g., Staphylococcus aureus, Staphylococcus epidermidis), Streptococcus (e.g., Streptococcus anginosus, Streptococcus mutans, Streptococcus oralis, Streptococcus pneumoniae, Streptococcus sobrinus, Streptococcus viridans), Torulopsis (e.g., Torulopsis glabrata), Treponema (e.g., Treponema denticola, Treponema refringens), Veillonella, Vibrio (e.g., Vibrio sputorum), Wolinella (e.g., Wolinella succinogenes), Yersinia (e.g., Yersinia enterocolitica) and any combination thereof.

Suitably, the gastrointestinal disease, disorder or condition is selected from the group consisting of candidiasis, celiac disease, Crohn's disease, diarrhoea, constipation, ulcerative colitis, food allergy, food intolerance, inflammatory bowel disease (IBD), irritable bowel syndrome (IBS), intestinal dysbiosis, metabolic syndrome, ulcers, digestion disorders, malabsorption syndromes, gastritis, enteritis, gastroesophageal reflux, eosinophilic gastroenteritis, infectious diarrhoea, collagenous colitis and lymphocytic colitis, diversion colitis, indeterminate colitis, nonsteroidal anti-inflammatory drug enteropathy, non-celiac gluten sensitivity, coeliac disease, acute self-limiting colitis, amoebic colitis, schistosomiasis, colon cancer, intestinal tuberculosis and any combination thereof.

In yet another aspect, the invention provides a method of promoting gastrointestinal health in a subject, said method including the step of administering to said subject a therapeutically effective amount of the orally administrable formulation described herein to thereby promote gastrointestinal health in the subject.

It will be understood that gastrointestinal health refers to the health and/or function of any or all of the component parts of a gastrointestinal tract of a subject, such as the oesophagus, stomach, small intestine, and the large intestine. The promotion or enhancement of gastrointestinal health of the subject may include, for example, improvements in gastrointestinal motility and gastric emptying, and/or reductions in constipation, heartburn, reflux, inflammation, flatulence, bloating and combinations thereof.

By “administration” or “administering” is meant the introduction of an orally administrable formulation (e.g., a formulation comprising, consisting or consisting essentially of a plurality of plant-based polyphenol sources and/or one or more components or derivatives thereof and one or more of a fibre source, a sweetening agent and a flavouring agent) into a subject by a chosen route, and in particular by the oral route.

The term “therapeutically effective amount” describes a quantity of a specified agent sufficient to achieve a desired effect in a subject being treated with that agent. For example, this can be the amount of the orally administrable formulation hereinbefore described to: (a) promote gastrointestinal health; (b) modulate microbial flora in at least a portion of the subject's gastrointestinal tract; and/or (c) reduce, alleviate and/or prevent a gastrointestinal disease, disorder or condition. In some embodiments, a “therapeutically effective amount” is sufficient to reduce or eliminate a symptom of such a disease, disorder or condition (e.g., diarrhoea). In other embodiments, a “therapeutically effective amount” is an amount sufficient to achieve a desired biological effect, for example an amount that is effective to decrease an inflammatory and/or immune response associated with a gastrointestinal disease, disorder or condition.

Ideally, a therapeutically effective amount of an agent is an amount sufficient to induce the desired result without causing a substantial cytotoxic effect in the subject. The effective amount of the orally administrable formulation useful for, for example, reducing, alleviating and/or preventing a gastrointestinal disease, disorder or condition will be dependent on the subject being treated, the type and severity of any associated disease, disorder and/or condition, and the manner of administration of the therapeutic composition.

In one embodiment, a given dosage of the orally administrable formulation is applied as a single application or a plurality of applications over a given time period (e.g., for as long as the subject requires treatment), where the dosing schedule is administered over a given time period, examples of which include hourly, daily, weekly, biweekly or monthly dosing schedules.

It would also be appreciated that one or more additional agents as are known in the art for reducing, alleviating and/or preventing a gastrointestinal disease, disorder and/or condition, or one or more symptoms associated therewith, may be administered to a subject in need thereof in addition to a therapeutically effective amount of the orally administrable formulation described herein. That is, one or more additional agents traditionally used for the treatment and/or prevention of a gastrointestinal disease, disorder and/or condition, such as an anti-inflammatory agent, an anti-diarrhoeal agent and the like, may be administered to a subject in addition to a therapeutically effective amount of the orally administrable formulation.

Any safe route of administration may be employed for providing a subject with a formulation of the present aspect. For example, oral, rectal, parenteral, sublingual, buccal, intravenous, intra-articular, intra-muscular, intra-dermal, subcutaneous, inhalational, intraocular, intraperitoneal, intracerebroventricular, transdermal, and the like may be employed. Most preferably, the formulation is orally administered.

Dosage forms include powder, tablets, dispersions, suspensions, injections, solutions, syrups, troches, capsules, suppositories, aerosols, transdermal patches, liquid drops, diluted in beverage, gum, confectionary, oral strips, gel, jelly, and the like. These dosage forms may also include injecting or implanting controlled releasing devices designed specifically for this purpose or other forms of implants modified to act additionally in this fashion.

The above formulations may be administered in a manner compatible with the dosage formulation, and in such amount as is pharmaceutically/therapeutically-effective. The dose administered to a subject, in the context of the present invention, should be sufficient to effect a beneficial response (e.g., an improvement in gastrointestinal health and/or a reduction or amelioration in symptoms of a gastrointestinal disease, disorder or condition) in a subject over an appropriate period of time. The quantity of the orally administrable formulation to be administered may depend on the subject to be treated, inclusive of the age, sex, weight and general health condition thereof, factors that will depend on the judgement of a practitioner of ordinary skill in the art.

In particular embodiments, administration of the orally administrable formulation modulates one or more species or genera of microbial flora in at least a portion of a gastrointestinal tract of the subject. The microbial flora may include one or more microorganisms of a genus selected from the group consisting of Achromobacter, Acidaminococcus, Acinetobacter, Actinomyces, Aeromonas, Aggregatibacter, Akkermansia, Alcaligenes, Alistipes, Anaerobiospirillum, Arachnia, Bacillus, Bacteroides, Bacterionema, Bifidobacterium, Buchnera, Butyriviberio, Campylobacter, Candida Capnocytophaga, Citrobacter, Clostridium, Corynebacterium, Eikenella, Enterobacter, Enterococcus, Escherichia, Eubacterium, Flavobacterium, Fusobacterium, Gordonia, Haemophilus, Lactobacillus, Leptotrichia, Methanobrevibacter, Morganella, Mycobacteria, Mycoplasma, Micrococcus, Neisseria, Parabacteroides, Peptococcus, Peptostreptococcus, Plesiomonas, Porphyromonas, Prevotella, Propionibacterium, Providencia, Pseudomonas, Ruminococcus, Rothia, Sarcina, Staphylococcus, Streptococcus, Torulopsis, Treponema, Veillonella, Vibrio, Wolinella, Yersinia and any combination thereof.

In yet a further aspect, the invention provides a method of modulating microbial flora in at least a portion of a gastrointestinal tract of a subject, said method including the step of administering to the subject the orally administrable formulation hereinbefore described in an amount effective to achieve said modulation.

As would be understood by the skilled person, the one or more microbial flora is deemed to be “modulated” when the relative or absolute number or concentration of the one or more microbial flora is increased/up regulated or decreased/down regulated when compared to a control or reference sample. By way of example, the control or reference sample may be from one or more subjects known to not have been administered the orally administrable formulation or it may be from said subject prior to being administered the orally administrable formulation. The control or reference sample may be a pooled, average or an individual sample. The modulation may be temporary or permanent.

In some embodiments, the number or concentration of the one or more microbial flora is increased if it is more than about 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500%, 600%, 700%, 800%, 900% or at least about 1000% greater than the number or concentration of the one or more microbial flora in a control or reference sample.

In some embodiments, the number or concentration of the one or more microbial flora is decreased if it is less than about 95%, 90%, 80%, 70%, 60%, 50%, 40%, 30%, 20% or 10%, or even less than about 5%, 4%, 3%, 2%, 1%, 0.5%, 0.1%, 0.01%, 0.001% or 0.0001% of the number or concentration of the one or more microbial flora in a control or reference sample.

Accordingly, administration of the orally administrable formulation may result in the reappearance of one or more normally occurring microbial flora that are no longer present or are decreased in quantity from the gastrointestinal system of the animal, and/or an increase in the number or concentration to levels comparable with or higher than those typically observed in healthy animals. Furthermore, the orally administrable formulation may produce a decrease in the number or concentration of one or more normally occurring and/or potentially pathogenic microbial flora in the gastrointestinal system of an animal. Additionally, the orally administrable formulation may inhibit or prevent variations in the microbial composition and/or microbial concentrations of the gastrointestinal flora of an animal.

In particular embodiments, the microbial flora include one or more microorganisms of a genus selected from the group consisting of Achromobacter, Acidaminococcus, Acinetobacter, Actinomyces, Aeromonas, Aggregatibacter, Akkermansia, Alcaligenes, Alistipes, Anaerobiospirillum, Arachnia, Bacillus, Bacteroides, Bacterionema, Bifidobacterium, Buchnera, Butyriviberio, Campylobacter, Candida Capnocytophaga, Citrobacter, Clostridium, Corynebacterium, Eikenella, Enterobacter, Enterococcus, Escherichia, Eubacterium, Flavobacterium, Fusobacterium, Gordonia, Haemophilus, Lactobacillus, Leptotrichia, Methanobrevibacter, Morganella, Mycobacteria, Mycoplasma, Micrococcus, Neisseria, Parabacteroides, Peptococcus, Peptostreptococcus, Plesiomonas, Porphyromonas, Prevotella, Propionibacterium, Providencia, Pseudomonas, Ruminococcus, Rothia, Sarcina, Staphylococcus, Streptococcus, Torulopsis, Treponema, Veillonella, Vibrio, Wolinella, Yersinia and any combination thereof. Preferably, administration of the orally administrable formulation increases the number or concentration of one or more microorganisms of, for example, the genus Akkermansia, Alistipes, Bacteroides, Parabacteroides, Porphyromonas and Prevotella and/or decreases the number or concentration of one or more microorganisms of, for example, the genus Candida, Clostridium and Lactobacillus.

In a related aspect, the invention provides a method of treating and/or preventing a gastrointestinal disease, disorder or condition in a subject, said method including the step of administering to said subject a therapeutically effective amount of the orally administrable formulation described herein, to thereby treat and/or prevent said gastrointestinal disease, disorder or condition in the subject.

As used herein, “treating”, “treat” or “treatment” refers to a therapeutic intervention, course of action or protocol that at least ameliorates a symptom of a gastrointestinal disease, disorder or condition after said disease, disorder or condition and/or its symptoms have at least started to develop. As used herein, “preventing”, “prevent” or “prevention” refers to therapeutic intervention, course of action or protocol initiated prior to the onset of a gastrointestinal disease, disorder or condition and/or a symptom thereof so as to prevent, inhibit or delay or development or progression of said disease, disorder or condition or the symptom. In this regard, a “prophylactic” treatment is a treatment administered to a subject who does not exhibit signs of a gastrointestinal disease, disorder or condition or exhibits only early signs for the purpose of decreasing the risk of developing a gastrointestinal disease, disorder or condition.

It will be appreciated that the gastrointestinal disease, disorder or condition may be any as are known in the art. In particular embodiments, the gastrointestinal disease, disorder or condition is selected from the group consisting of candidiasis, celiac disease, Crohn's disease, diarrhoea, constipation, ulcerative colitis, food allergy, food intolerance, inflammatory bowel disease (IBD), irritable bowel syndrome (IBS), intestinal dysbiosis, metabolic syndrome, ulcers, digestion disorders, malabsorption syndromes, gastritis, enteritis, gastroesophageal reflux, eosinophilic gastroenteritis, infectious diarrhoea, collagenous colitis and lymphocytic colitis, diversion colitis, indeterminate colitis, nonsteroidal anti-inflammatory drug enteropathy, non-celiac gluten sensitivity, coeliac disease, acute self-limiting colitis, amoebic colitis, schistosomiasis, colon cancer, intestinal tuberculosis and any combination thereof.

The term “subject”, as used herein, includes both human and veterinary subjects. For example, administration to a subject can include administration to a human subject or a veterinary subject. Preferably, the subject is a human. However, therapeutic uses according to the invention may also be applicable to mammals such as domestic and companion animals, performance animals such as horses, livestock, and laboratory animals.

All computer programs, algorithms, patent and scientific literature referred to herein is incorporated herein by reference.

So that the present invention may be more readily understood and put into practical effect, the skilled person is referred to the following non-limiting examples.

### Example 1

Example 1 provides an embodiment of the orally administrable formulation as a “modbiotic” powder. This powdered product is designed to be consumed after the addition to a liquid beverage, such as water or juice. A lower dose of 2.5 to 10 g per day may be administered to maintain or promote gastrointestinal health or a higher dose of 5 to 30 g per day for a shorter period of time may be administered to modulate the gut microbiome.

| Ingredient | Amount | |
|---|---|---|
| daikon radish powder | 50 | mg |
| kale powder | 50 | mg |
| Broccoli sprout powder | 100 | mg |
| Barley powder | 50 | mg |
| Acai berry powder | 50 | mg |
| Larix Heartwood powder | 750 | mg |
| Hibiscus sabdarifa powder | 750 | mg |
| Cacao powder | 300 | mg |
| Prunus serotina powder | 100 | mg |
| nutmeg powder | 150 | mg |
| ginger powder | 100 | mg |
| cinnamon powder | 150 | mg |
| Green banana flour | 500 | mg |
| Schisandra chinensis powder | 150 | mg |
| pomegranate peel powder | 150 | mg |
| rosemary powder | 100 | mg |
| Glucosamine Hydrochloride | 500 | mg |
| apple peel powder | 100 | mg |
| cranberry powder | 100 | mg |
| stevia powder | 3 | mg |
| rice bran extract available as an | 50 | mg |
| alternative silicon excipient | | |

### Example 2

Example 2 provides an embodiment of the orally administrable formulation as a “modbiotic” capsule. The encapsulated orally administrable formulation is designed to be administered at a dose of 1 to 12 capsules daily. A lower dose of 1 capsule 1-3 times per day may be administered to maintain or promote gastrointestinal health or a higher dose of 1 to 3 capsules taken 3 to 4 times per day may be administered to modulate the gut microbiome.

| | |
|---|---|
| Ingredients | |
| Ganoderma lucidium (Reishi) powder | 10-50% |
| orgen-C (Vitamin C) | 10-50% |
| Turmeric powder | 5-25% |
| Boswellia serrata (Frankincense) | 5-25% |
| Commiphora myrrha | 5-25% |
| Artemisia annua | 5-25% |
| dose/serve size | |
| 2 to 6 capsules daily | |

### Example 3

Example 3 provides an embodiment of the orally administrable formulation as a “modbiotic” food product, such as a bar, biscuit, granola, cereal, snacks, muffins, etc. and is based on high polyphenols and microbiome modulating fibres and flours.

### Ingredients

1-10% “Modbiotic” polyphenol plant powder or blend consisting of one or more of the following ingredients:
daikon radish
israeli mustard
watercress
kale
beetroot juice powder
barley sprout powder
acai berry
Ganoderma lucidium (Reishi)

### Larix Heartwood

Hibiscus sabdarifa
Boswellia serrata
cacao
cacao husk
Prunus serotina
nutmeg
ginger powder
cinnamon extract
Broccoli sprout
Schisandra chinensis
Artemisia annua
Pseudowintera colorata
graviola
cranberry
pomegranate
citrus peels (pomegranate, grapefruit, lemon, orange, bergamot)
rosemary
nut skins (hazelnut, peanut, pistachio, almond etc.)
apple peel
20-70% Flour or blend including one or more of the following ingredients: konjac flour, slippery elm, psyllium husk, chick pea flour and/or fibre, green banana flour, faba bean powder, mung bean powder, almond, hazelnut, peanut, sunflower, linseed, quinoa, teff, freekeh, oat, wheat, spelt, any legume meal, any seed meal, and/or any other grain flours)
1-40% fats/oil or blend including animal fats, margarine, vegetable oil, nut oils and seed oils
0.25-30% liquid or blend including water, milk, nut juice, fruit juice, vegetable juice, honeys, syrups, or liquid sweeteners and fats
10-60% nutritive sweeteners such as but not exclusively; sugars, honeys, syrups, malts, molasses and/or 0.25-10% non-nutritive sweeteners such as but not exclusively; stevia, xylitol, erythritol, sucralose, aspartame, saccharin, acesulfame-K, thaumatin, kemfe, katemfe, magrosides or combinations thereof.

### Example 4

Example 4 provides an embodiment of the orally administrable formulation as a “modbiotic” food product, such as a lolly, gummy, gel bar and is based on high polyphenols in a gelatine base

(a) 1-10% “Modbiotic” polyphenol plant powder or blend consisting of one or more of the following ingredients:
daikon radish
israeli mustard
watercress
kale
beetroot juice powder
barley sprout powder
acai berry
Ganoderma lucidium (Reishi)

### Larix Heartwood

Hibiscus sabdarifa
Boswellia serrata
cacao
cacao husk
Prunus serotina
nutmeg
ginger powder
cinnamon extract
Broccoli sprout
Schisandra chinensis
Artemisia annua
Pseudowintera colorata
graviola
cranberry
pomegranate
citrus peels (pomegranate, grapefruit, lemon, orange, bergamot)
rosemary
nut skins (e.g., hazelnut, peanut, pistachio, almond etc.)
apple peel
(b) one or more gelling agents;
(c) one or more flavouring agents;
(d) one or more sweetening agents; and
(e) one or more organic acids;

PRODUCT 1 - 10% “modbiotic blend” added
to Marshmallow collagen/gelatine Bar base

| | | |
|---|---|---|
| A: | DAVIS ® J3 Agar | 0.47% |
| | Water | 9.38% |
| B: | Hydrolysed collagen | 46.97% |
| | Water | 9.38% |
| C: | Erythritol | 9.28% |
| | Gelatine | 6.84% |
| D: | Polydextrose | 10.03% |
| | Water | 3.75% |
| E: | Flavours | 0.70% |
| | Colours | 0.09% |
| | Stevia | 0.05% |
| | Malic Acid | 0.95% |
| | Ascorbic Acid | 1.90% |
| | Tryptophan | 0.02% |
| | Cultured Dextrose | 1.88% |

### PROCESS

1. Cook part A to boiling and hold for 1 minute, once partially thickened add part C and mix well and cook until. Mix part B together and heat and hold at 70 degrees while other ingredients cook. Add part D to part A and C mixture and continue to cook. Once syrup reaches a specific solids content, add to mixture B and mix well. Add in components of part E and incorporate well, once incorporated aerate the mixture to a specific gravity/density of 0.5-0.7.
Keep the mixture at around 70° C. in a hopper or holding tank until ready to deposit. 2. Deposit the syrup into oiled silicone or plastic moulds. Dry the bars at room temperature for 1-2 hrs, remove bars from moulds and pack.

### Example 5

Example 5 provides an embodiment of the orally administrable formulation as a “modbiotic” food product, such as an ice-cream, frozen custard and is based on high polyphenols in a gelatine base

As ice cream:

### A:

### Cream 27.20%

### Water 26.75%

### Milk 24.50%

### B:

sweetener 10%
Powdered eggs 1.00%

### Gelatine 15.35%

Flavour as required
Process: Heat A to 75° C. Hold at this temperate for 1 minute. Allow to cool to 67° C. and add B. Blend until smooth. Chill for 4 hours then churn.

1. An orally administrable formulation for promoting gastrointestinal health comprising, consisting or consisting essentially of:
(i) a plurality of plant-based polyphenol sources and/or one or more components or derivatives thereof; and
(ii) one or more of a fibre source, a sweetening agent and/or a flavouring agent.

2. The formulation of claim 1, wherein the plant-based polyphenol source is selected from the group consisting of Raphanus sativus (daikon radish), Brassica oleracea spp., Hordeum vulgare (barley), Euterpe oleracea (acai), Larix spp. heartwood, Hibiscus sabdarifa (rosella), Theobroma cacao, Prunus serotina (black cherry), Myristica fragrans (nutmeg), Zingiber officinale (ginger), Allium cepa (onions), Allium sativum (garlic), Cinnamomum verum or other cinnamon species, Musa spp. (green banana), Schisandra chinensis, Punica granatum (pomegranate), Rosmarinus officinalis (rosemary), Malus spp. (apple), Vaccinium spp. (cranberry), berberine, Ilex paraguariensis (yerba mate), Coffea spp., Ganoderma spp., Lentinula edodes (shiitake), Curcuma longa (turmeric), Boswellia spp. (frankincense), Artemisia spp., Matricaria chamomilla (chamomile), Rumex crispus (yellow dock), Mahonia aquifolium (oregon grape), Hydrastis canadensis (goldenseal), Calendula spp., Vicia faba (faba bean), Vigna radiata (mungbean), Cicer arietinum (chick pea), Pseudowintera colorata, graviola (soursop), Pimpinella anisum (anise), Lycium barbarum (goji berry), Lycium chinense (goji berry), Prunus spp. (cherry), Beta vulgaris (beetroot), Commiphora spp. (myrrh), Salvadora persica (Israeli mustard) a nut, a herb, a fruit extract, a vegetable extract and any combination thereof.

3. The formulation of claim 1, wherein one or more of the plant-based polyphenol sources and/or one or more components or derivatives thereof, includes one or more of an extract thereof, a skin portion, a peel portion, a seed portion, a leaf portion, a sprout portion, a juice portion, a husk portion, a root portion and a pulp portion.

4. The formulation of claim 1, wherein the fibre source is selected from the group consisting of a konjac fibre source or flour, a psyllium fibre source, a mucopolysaccharide, inulin, a sugarcane fibre source, a chick pea fibre source, a green banana fibre source, a faba bean fibre source, a mung bean fibre source, a nut meal, a legume meal, a seed meal, a grain flour, a husk flour, a bran flour and any combination thereof.

5. The formulation of claim 1, wherein the formulation is substantially free of probiotic microorganisms.

6. The formulation of claim 1, wherein the formulation comprises from about 40 wt % to about 95 wt % of the plant-based polyphenol sources.

7. The formulation of claim 1, wherein the formulation comprises from about 1 wt % to about 25 wt % of the fibre source.

8. The formulation of claim 1, wherein the formulation comprises from about 0.1 wt % to about 5 wt % of the sweetening agent.

9. The formulation of claim 1, wherein the formulation comprises from about 0.1 wt % to about 5 wt % of the flavouring agent.

10. The formulation of claim 1, further comprising one or more vitamins and/or minerals.

11. The formulation of claim 1, wherein the formulation is in the form of a food product.

12. A method of producing an orally administrable formulation, including the step of combining a plurality of plant-based polyphenol sources and/or one or more components or derivatives thereof with a fibre source, a sweetening agent and/or a flavouring agent to thereby produce the orally administrable formulation.

13. (canceled)

14. The formulation of claim 1, for use in:
(i) promoting gastrointestinal health;
(ii) modulating microbial flora in at least a portion of a gastrointestinal tract; and/or
(iii) the therapeutic and/or prophylactic treatment of a gastrointestinal disease, disorder or condition,
in a subject.

15. A method of promoting gastrointestinal health in a subject, said method including the step of administering to said subject a therapeutically effective amount of an orally administrable formulation comprising, consisting or consisting essentially of:
(i) a plurality of plant-based polyphenol sources and/or one or more components or derivatives thereof; and
(ii) one or more of a fibre source, a sweetening agent and/or a flavouring agent,
to thereby promote gastrointestinal health in the subject.

16. The method of claim 15, wherein administration of the orally administrable formulation modulates one or more species or genera of microbial flora in at least a portion of a gastrointestinal tract of the subject.

17. A method of modulating microbial flora in at least a portion of a gastrointestinal tract of a subject, said method including the step of administering to the subject an orally administrable formulation comprising, consisting or consisting essentially of:
(i) a plurality of plant-based polyphenol sources and/or one or more components or derivatives thereof; and
(ii) one or more of a fibre source, a sweetening agent and/or a flavouring agent, in an amount effective to achieve said modulation.

18. The method of claim 16 or 17, wherein the microbial flora include one or more microorganisms of a genus selected from the group consisting of Achromobacter, Acidaminococcus, Acinetobacter, Actinomyces, Aeromonas, Aggregatibacter, Akkermansia, Alcaligenes, Alistipes, Anaerobiospirillum, Arachnia, Bacillus, Bacteroides, Bacterionema, Bifidobacterium, Buchnera, Butyriviberio, Campylobacter, Candida Capnocytophaga, Citrobacter, Clostridium, Corynebacterium, Eikenella, Enterobacter, Enterococcus, Escherichia, Eubacterium, Flavobacterium, Fusobacterium, Gordonia, Haemophilus, Lactobacillus, Leptotrichia, Methanobrevibacter, Morganella, Mycobacteria, Mycoplasma, Micrococcus, Neisseria, Parabacteroides, Peptococcus, Peptostreptococcus, Plesiomonas, Porphyromonas, Prevotella, Propionibacterium, Providencia, Pseudomonas, Ruminococcus, Rothia, Sarcina, Staphylococcus, Streptococcus, Torulopsis, Treponema, Veillonella, Vibrio, Wolinella, Yersinia and any combination thereof.

19. A method of treating and/or preventing a gastrointestinal disease, disorder or condition in a subject, said method including the step of administering to said subject a therapeutically effective amount of an orally administrable formulation of comprising, consisting or consisting essentially of:
(i) a plurality of plant-based polyphenol sources and/or one or more components or derivatives thereof, and
(ii) one or more of a fibre source, a sweetening agent and/or a flavouring agent, to thereby treat and/or prevent said gastrointestinal disease, disorder or condition in the subject.

20. The method of claim 19, wherein the gastrointestinal disease, disorder or condition is selected from the group consisting of candidiasis, celiac disease, Crohn's disease, diarrhoea, constipation, ulcerative colitis, food allergy, food intolerance, inflammatory bowel disease (IBD), irritable bowel syndrome (IBS), intestinal dysbiosis, metabolic syndrome, ulcers, digestion disorders, malabsorption syndromes, gastritis, enteritis, gastroesophageal reflux, eosinophilic gastroenteritis, infectious diarrhoea, collagenous colitis and lymphocytic colitis, diversion colitis, indeterminate colitis, nonsteroidal anti-inflammatory drug enteropathy, non-celiac gluten sensitivity, coeliac disease, acute self-limiting colitis, amoebic colitis, schistosomiasis, colon cancer, intestinal tuberculosis and any combination thereof.

21. (canceled)

22. The method of claim 17, wherein the microbial flora include one or more microorganisms of a genus selected from the group consisting of Achromobacter, Acidaminococcus, Acinetobacter, Actinomyces, Aeromonas, Aggregatibacter, Akkermansia, Alcaligenes, Alistipes, Anaerobiospirillum, Arachnia, Bacillus, Bacteroides, Bacterionema, Bifidobacterium, Buchnera, Butyriviberio, Campylobacter, Candida Capnocytophaga, Citrobacter, Clostridium, Corynebacterium, Eikenella, Enterobacter, Enterococcus, Escherichia, Eubacterium, Flavobacterium, Fusobacterium, Gordonia, Haemophilus, Lactobacillus, Leptotrichia, Methanobrevibacter, Morganella, Mycobacteria, Mycoplasma, Micrococcus, Neisseria, Parabacteroides, Peptococcus, Peptostreptococcus, Plesiomonas, Porphyromonas, Prevotella, Propionibacterium, Providencia, Pseudomonas, Ruminococcus, Rothia, Sarcina, Staphylococcus, Streptococcus, Torulopsis, Treponema, Veillonella, Vibrio, Wolinella, Yersinia and any combination thereof.
